(19) United States
(12) Patent Application Publication
(10) Pub. No.: US 2024/0252516 A1
(43) Pub. Date: Aug. 1, 2024

(54) COMBINATION BPH THERAPY WITH FEXAPOTIDE TRIFLUTATE, A 5-ARI, AND AN ALPHA BLOCKER

(71) Applicant: Nymox Corporation, Nassau (BS)

(72) Inventor: Paul Averback, Nassau (BS)

(21) Appl. No.: 18/161,922

(22) Filed: Jan. 31, 2023

(51) Int. Cl. A61K 31/58; A61K 31/18; A61K 38/10; A61K 45/06; A61P 13/08

(52) U.S. Cl. CPC A61K 31/58; A61K 31/18; A61K 38/10; A61K 45/06; A61P 13/08

(57) ABSTRACT

Disclosed are methods of treating benign prostatic hyperplasia that include administering a composition containing at least Fexapotide Triflutate (FT), and subsequently administering one or more compositions containing a 5 α-reductase inhibitor (5-ARI) and an alpha1-adrenergic receptor blocker (alpha blocker). The methods provide dramatically improved mean IPSS values, when compared to the average mean IPSS of the individual actives alone, or when compared to the average mean IPSS of (a) the composition containing FT; and (b) the combination of 5-ARI and alpha blocker.

### BACKGROUND

### 1. Field of the Embodiments

The embodiments include compositions and methods for treating benign prostatic hyperplasia using combination therapy that includes administering compositions containing fexapotide triflutate (“FT”), and separately administering one or more composition(s) comprising a 5 α-reductase inhibitor (5-ARI) and an alpha1-adrenergic receptor blocker (alpha blocker). The combination BPH therapy is more effective in improving International Prostate Symptom Scores (IPSS) than other combination BPH therapies including combinations of FT and 5-ARIs, combinations of FT and alpha blockers, combinations of FT and PDE5 inhibitors, and combinations of 5-ARIs and alpha blockers.

### 2. Sequence Listing

The instant application contains a Sequence Listing which has been submitted electronically in XML format and is hereby incorporated by reference in its entirety. Said XML copy, created on Apr. 13, 2023, is name 063307-0571350.XML and is 4,096 bytes in size.

### 3. Description of Related Art

The essence of many medical treatments and procedures involves the removal or destruction of harmful or unwanted tissue. Examples of such treatments include the surgical removal of cancerous or pre-cancerous growths, the destruction of metastatic tumors through chemotherapy, and the reduction of glandular (e.g. prostate) hyperplasia. Other examples include the removal of unwanted facial hair, the removal of warts, and the removal of unwanted fatty tissue.

Benign prostatic hyperplasia (BPH) is common in older men, with symptoms that impact quality of life, including interference with activities and perception of well-being. BPH can be progressive, with risk of urinary retention, infections, bladder calculi, and renal failure. Although many men with mild to moderate symptoms do well without intervention, bothersome symptoms and complications can progress in others, leading to medical therapy or surgery.

Benign Prostatic Hyperplasia (BPH) is a histologic diagnosis that refers to the nonmalignant proliferation of smooth muscle and epithelial cells of the prostate. Lee C, et al., “Intrinsic and extrinsic factors controlling benign prostatic growth,” Prostate, 1997; 31:131-138; Auffenberg G B, et al., “Established medical therapy for benign prostatic hyperplasia,” Urol Clin North Am,. 2009; 36:443-459. The exact etiology is unknown. The progression of BPH can lead to benign prostatic enlargement (BPE), which is determined by the size of the prostate (pathologic). Approximately 50% of men with histologic BPH develop BPE. BPE may eventually cause bladder outlet obstruction (BOO), which is also termed benign prostatic obstruction (BPO) if associated with BPE. BOO and BPO are determined with urodynamic measures.

Some agents known to have the ability to destroy and hence either facilitate the removal of or inhibit the further growth of harmful or unwanted cells and tissue are disclosed in U.S. Pat. Nos. 11,298,400; 11,278,588; 10,532,081; 9,243,035; 8,716,247; 8,569,446; 8,293,703; 8,067,378; 7,745,572; 7,408,021; 7,317,077; 7,241,738; and 7,192,929, the disclosures of each of which are incorporated by reference herein in their entirety. Additional agents known to have the ability to destroy and hence either facilitate the removal of or inhibit the further growth of harmful or unwanted cells and tissue are disclosed in U.S. Patent Application Publication Nos. 2020/0360466; 2020/0061150; 2017/0020957; 2016/0215031; 2016/0361380; 2007/0237780 (now abandoned), the disclosures of each of which are incorporated by reference herein in their entirety.

One of the agents disclosed in these documents is fexapotide triflutate, or FT. FT has been shown to reduce prostate glandular cells. In controlled clinical studies for treating BPH, and some of its symptoms, the overall improvements with FT were greater than placebo treatments after long-term observations.

In the prostate as well as in many other tissues, testosterone is irreversibly converted by 5α-reductase into the more potent androgen dihydrotestosterone (Bruchovsky and Wilson, J. Biol. Chem. 243: 2012-2021, 1968; Wilson, Handbook of Physiology 5 (section 7), pp. 491-508, 1975). Inhibitors of 5 α-reductase have been found to inhibit prostatic growth (Brooks et al., Endocrinology 109: 830, 1981; Brooks et al., Proc. Soc. Exp. Biol. Med. 169: 67, 1982; Brooks et al., Prostate 3: 35, 1982; Wenderoth et al., Endocrinology 113,569-573, 1983; McConnell et al., J. Urol. 141: 239A, 1989; Stoner, E., Lecture on the role of 5 alpha-reductase inhibitor in benign prostatic hypertropy, 84th AUA Annual Meeting, Dallas, May 8, 1989.).

The inhibitory effect of the 5 α-reductase inhibitor Merck L 652,931 on prostatic and seminal vesicle development in the prepubertal rat was described in Proc. 71st Annual Meeting of Endocr. Soc. abst. #1165, p. 314, 1989. The inhibitory effect of MK-906 on dihydrotestosterone formation in men has been described in men by Gormley et al., in Proc. 71st Annual Meeting of Endocr. Soc., abst. #1225, p. 329, 1989; Imperato-McGinley et al., in Proc. 71st Annual Meeting of Endocr. Soc., abst. #1639, p. 432, 1989; Geller and Franson, in Proc. 71st Annual Meeting of Endocr. Soc., abst. #1640, p. 432, 1989 and Tenover et al., in Proc. 71st Annual Meeting of Endocr. Soc., abst. #583, p. 169, 1989. The activity of the 5 α-reductase inhibitors N,N-diethyl-4-methyl-3-oxo-4-aza-5.alpha.-androstane-17.beta.-carboxamide (4-MA) and 6-methylene-4-pregnene-3,20-dione (LY 207320) has been described by Toomey et al., Proc. 71st Annual Meeting of Endocr. Soc., abst. #1226, p. 329, 1989.

In addition to the well-known effect of androgens on prostatic growth, there are many studies which show that estrogens play also a role in proliferation of the prostate (Walsh and Wilson, J. Clin. Invest. 57: 1093-1097, 1976; Robinette et al., Invest. Urol. 15: 425-432, 1978; Moore et al., J. Clin. Invest. 63: 351-257, 1979). Moreover, estrogens have been shown to enhance androgen-induced prostatic growth in the dog (Walsh and Wilson, J. Clin. Invest. 57: 1093-1097, 1976; Jacobi et al., Endocrinology 102: 1748-1755, 1978; Tunn et al., Urol. Int. 35: 125-140, 1980). A possible explanation of this enhancing effect of estrogen on androgen-induced prostate growth, is the observation that 17β-estradiol has been shown to increase androgen binding in the dog prostate (Moore et al., J. Clin. Invest. 63: 351-357, 1979).

The antiestrogen Tamoxifen has been shown to improve steroid-induced benign prostatic hyperplasia in the dog (Funke et al., Acta Endocrinol. 100: 462-472, 1982). Administration of the antiestrogen Tamoxifen in association with the steroidal antiandrogen cyproterone acetate in patients suffering from benign prostatic hyperplasia showed beneficial effects on the symptoms of the disease (Di Silverio et al., in Ipertrofia Prostatica Benigna (F. Di Silverio, F. Neumann and M. Tannenbaum, eds), Excerpta Medica, pp. 117-125, 1986). In U.S. Pat. No. 4,310,523, it is proposed that a combination of an antiandrogen and an antiestrogen is effective for the prophylaxis and/or therapy of benign prostatic hyperplasia. Tamoxifen, however, has intrinsic estrogenic activity which limits its effectiveness.

Estrogen formation resulting from aromatization of androgens, occurs at several sites. In the male, aromatization of androgens has been demonstrated in the testis, adipose and muscle tissue, skin, liver, brain and prostate (Schweikert et al., J. Clin. Endocrinol. Metab. 40: 413-417, 1975; Folker and James, J. Steroid Biochem. 49: 687-690, 1983; Longcope et al., J. Clin. Endocrinol. Metab. 46: 146-152, 1978; Lacoste and Labrie, unpublished data; Stone et al., The Prostate 9: 311-318, 1986; Stone et al., Urol. Res. 15: 165-167, 1987). There is evidence for an increased production of estrogens in the prostatic tissue of benign prostatic hyperplasia patients (Stone et al., The Prostate 9: 311-318, 1986). Such data indicate that the local formation of estrogens may play a crucial role in stimulating prostatic growth in excess of the action predicted by circulating estrogens.

U.S. Pat. No. 4,472,382 discloses treatment of BPH with an antiandrogen and certain peptides which act as LH-RH agonists. U.S. Pat. No. 4,596,797 discloses aromatase inhibitors as a method of prophylaxis and/or treatment of prostatic hyperplasia. U.S. Pat. No. 4,760,053 describes a treatment of certain cancers which combines an LHRH agonist with an antiandrogen and/or an antiestrogen and/or at least one inhibitor of sex steroid biosynthesis. U.S. Pat. No. 4,775,660 discloses a method of treating breast cancer with a combination therapy which may include surgical or chemical prevention of ovarian secretions and administering an antiandrogen and an antiestrogen.

U.S. Pat. No. 4,659,695 discloses a method of treatment of prostate cancer in susceptible male animals including humans whose testicular hormonal secretions are blocked by surgical or chemical means, e.g. by use of an LHRH agonist, which comprises administering an antiandrogen, e.g. flutamide, in association with at least one inhibitor of sex steroid biosynthesis, e.g. aminoglutethimide and/or ketoconazole. The disclosures of each of the above-mentioned patents (U.S. Pat. Nos. 4,472,382, 4,596,797, 4,760,053, 4,775,660, and 4,659,695) are incorporated by reference herein in their entireties.

BPH is caused by increased activity of both androgens and estrogens. Because of such a dual etiology of BPH, proposed hormonal therapies have been less than satisfactory and have all been unpredictable while, frequently, causing unacceptable side-effects. Moreover, the prior art treatment seldomly resulted in a decrease in prostatic volume above about 20 to 30% with inconsistent effects on the symptomatology (Scott and Wade, J. Urol. 101: 81-85, 1969; Caine et al., J. Urol. 114: 564-568, 1975; Peters and Walsh, New Engl. J. Med. 317: 599-604, 1987; Gabrilove et al., J. Clin. Endocrinol. Metab. 64: 1331-1333, 1987; Stone et al., J. Urol. 141: 240A, 1989; Clejan et al., J. Urol. 141: 534A, 1989; Stoner, E., Lecture on the role of 5 α-reductase inhibitor in benign prostatic hypertrophy, 84th AUA Annual Meeting, Dallas, May 8, 1989.

The elucidation of the mechanism summarized above has resulted in the recent development of effective agents to control, and in many cases reverse, the advance of BPH. In the forefront of these agents is Merck & Co., Inc.s' product PROSCAR® (finasteride). The effect of this compound is to inhibit the enzyme testosterone 5α reductase, which converts testosterone into 5α-dihydrotesterone, resulting in a reduced rate of prostatic enlargement, and often reduction in prostatic mass.

The development of such agents as PROSCAR® bodes well for the long-term control of BPH. However, as may be appreciated from the lengthy development of the syndrome, its reversal also is not immediate. In the interim, those males suffering with BPH continue to suffer, and may in fact lose hope that the agents are working sufficiently rapidly.

In response to this problem, one solution is to identify pharmaceutically active compounds which complement slower-acting therapeutics by providing acute relief. Agents that induce relaxation of the lower urinary tract tissue, by binding to alpha 1 adrenergic receptors, thus reducing the increased adrenergic tone due to the disease, would be good candidates for this activity. Thus, one such agent is alfuzosin, which is reported in EP 0 204597 to induce urination in cases of prostatic hyperplasia. Likewise, in WO 92/00073, the selective ability of the R(+) enantiomer of terazosin to bind to adrenergic receptors of the alpha1 subtype was reported. In addition, in WO 92/16213, combinations of S α-reductase inhibitory compounds and alpha1-adrenergic receptor blockers (terazosin, doxazosin, prazosin, bunazosin, indoramin, alfulzosin) were disclosed. However, no information as to the alpha 1d, alpha 1b, or alpha 1a subtype specificity of these compounds was provided as this data and its relevancy to the treatment of BPH was not known. Current therapy for BPH uses existing non-selective alpha 1 antagonists such as prazosin (Minipress, Pfizer), Terazosin (Hytrin, Abbott) or doxazosin mesylate (Cardura, Pfizer). These non-selective antagonists suffer from side effects related to antagonism of the alpha 1d and alpha 1b receptors in the peripheral vasculature, e.g., hypotension and syncope.

The cloning of the human alpha 1a adrenergic receptor (ATCC CRL 11140) and the use of a screening assay utilizing the cloned human alpha 1a receptor enables identification of compounds which specifically interact with the human alpha 1a adrenergic receptor. [PCT International Application Publication Nos. WO94/08040, published Apr. 14, 1994 and WO94/10989, published May 26, 1994]

WO 96/14846, published May 23, 1996, discloses a broad genus of dihydropyrimidine compounds and proposes their use as selective antagonists for human alpha 1a receptors. Compounds were assayed using cloned human alpha adrenergic receptors, and certain of the compounds so assayed were disclosed to be selective alpha 1a antagonists.

Combination BPH therapies are known in the art. To be effective, a combination therapy must at least be more effective than either active, or combinations of actives alone. One way to measure the effectiveness of combination therapies is to determine the improvement relative to the average improvement of the individual therapies, where the improvement for each treatment is determined relative to a control. For a triple combination, the effectiveness can be measured by determining the improvement relative to the average improvement of one of the actives and a combination of the other two actives (e.g., the average of the improvements of (a) 1 alone, and (b) the improvement of the combination of 2 and 3). It also was known that combinations of medications, even those having the same or similar mechanism of action, are rarely if ever, purely additive, when used in a clinical setting. In other words, a person having ordinary skill in the art would not have expected a combination of BPH medications to have a purely additive effect where the effects of each individual medication can simply be added together to provide a combined effect.

One known combination BPH therapy is a combination of dutasteride and tamsulosin, which was studied in the CombAT trial. In the CombAT trial, which is discussed in Miller, et al., “Combination therapy with dutasteride and tamsulosin for the treatment of symptomatic enlarged prostate,” Clin. Interv. Aging, 2009; 4:251-258, the mean decrease in IPSS from baseline after 2 years was 6.2 for BPH patients (Miller at pg. 8). In the CombAT trial, the improvement in IPSS for tamsulosin alone was 4.3, and the improvement in IPSS for dutasteride alone was 4.9. Thus, the combination did not produce an additive effect, and was about 34.7% better than the average improvement of each individual therapy. The combination of tamsulosin and dutasteride was approved by the FDA in 2010, and is sold under the brand name JALYN®.

Another known combination BPH therapy is a combination of doxazosin and finasteride, which is discussed in McConnell, et al., “The long-term effect of doxazosin, finasteride, and combination therapy on the clinical progression of benign prostatic hyperplasia,” N Engl J. Med., Vol. 349, pp. 2387-2398 (2003). McConnell discloses on pg. 2393 that the long term (4 year) mean IPSS improvement for doxazosin alone was 6.6, for finasteride alone was 5.6, and for the combination was 7.4. The average mean IPSS improvement for each active alone therefore is about 6.1, and thus the combination of doxazosin and finasteride provided an improvement of about 21.3%, when compared to the average mean IPSS improvement of both actives alone. As seen from the above, neither of the known combination therapies provides an improvement that is the additive effect of each therapy alone.

There exists a need for combinations of compositions and treatments that can improve the symptoms of BPH, as measured by the IPSS, that include combinations of BPH actives that provide greater improvement than known combinations.

Throughout this description, including the foregoing description of related art, any and all publicly available documents described herein, including any and all U.S. patent published patent applications, are specifically incorporated by reference herein in their entirety. The foregoing description of related art is not intended in any way as an admission that any of the documents described therein, including pending U.S. patent applications, are prior art to the present disclosure. Moreover, the description herein of any disadvantages associated with the described products, methods, and/or apparatus, is not intended to limit the embodiments. Indeed, aspects of the embodiments may include certain features of the described products, methods, and/or apparatus without suffering from their described disadvantages.

### SUMMARY OF THE EMBODIMENTS

There remains a need in the art for new, less toxic, and more effective treatments for improving the symptoms of BPH. The embodiments satisfy these needs.

This disclosure is premised in part on the discovery that certain peptides, including a specific peptide described by the amino acid sequence lle-Asp-Gln-Gln-Val-Leu-Ser-Arg-Ile-Lys-Leu-Glu-lle-Lys-Arg-Cys-Leu, (Fexapotide Triflutate or “FT”), when combined with at least one 5 α-reductase inhibitor (5-ARI) and at least one alpha1-adrenergic receptor blocker (alpha blocker) provide an unexpectedly superior improvement in treating BPH, as measured by a decrease in mean IPSS scores, when compared to the improvement found from either therapy alone, the average improvement of the three therapies, and the improvement from the average of the improvement from FT and the improvement from the combination of at least one 5-ARI and at least one alpha blocker.

The embodiments also include a combination of BPH therapies that includes a composition comprising FT and a pharmaceutically acceptable carrier, a composition comprising at least one 5-ARI and a pharmaceutically acceptable carrier, and a composition comprising at least one alpha blocker and a pharmaceutically acceptable carrier. Alternatively, the 5-ARI and alpha blocker may be combined in a single composition, also including a pharmaceutically acceptable carrier. In one embodiment, the composition comprising FT is an injectable composition that is administered once, or administered more than once with a period of time between each administration of from about 1 month to about 3 years, and the compositions comprising the 5-ARI and alpha blocker, or a composition comprising a combination of a 5-ARI and an alpha blocker, are compositions administered orally and taken daily, or intermittently (e.g., every other day, every three days, every four days, every day for 3 days, then one day off, and the like) over a period of time.

The composition comprising FT can be administered intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transrectally, transperitoneally, transdermally, via an aerosol, infusion, bolus injection, implantation device, sustained release system etc. Alternatively, FT can be expressed in vivo by administering a gene that expresses FT, by administering a vaccine that induces such production or by introducing cells, bacteria or viruses that express FT in vivo, because of genetic modification or otherwise. In an embodiment, the compositions comprising the 5-ARI and alpha blocker, or a single composition comprising a combination of a 5-ARI and an alpha blocker, can be administered together or separately from the composition comprising FT or the agent that expresses FT in vivo.

Another embodiment includes a method of treating BPH by administering a first composition comprising a therapeutically effective amount of FT; and subsequently orally administering a combination of a 5-ARI and an alpha blocker present in a single oral composition, or present in two separate oral compositions. In accordance with this method, the relative mean IPSS improvement of the combination may be from about 40% to about 100%, when compared to the average median IPSS improvement of all three actives alone, and the relative mean IPSS improvement of the combination may be from about 45% to about 400%, when compared to the average mean IPSS improvement of (a) FT alone and (b) the combination of a 5-ARI and an alpha blocker.

Both the foregoing general description and the following detailed description are exemplary and explanatory and are intended to provide further explanation of the embodiments as claimed. Other objects, advantages, and features will be readily apparent to those skilled in the art from the following detailed description of the embodiments.

### DETAILED DESCRIPTION OF THE PREFERRED EMBODIMENTS

Before the embodiments are described, it is understood that this invention is not limited to the particular methodology, protocols, cell lines, vectors, and reagents described, as these may vary. It also is to be understood that the terminology used herein is for the purpose of describing particular embodiments only, and is not intended to limit the scope of the present embodiments which will be limited only by the appended claims.

Terms and phrases used herein are defined as set forth below unless otherwise specified. Throughout this description, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Amino acids and amino acid residues described herein may be referred to according to the accepted one or three-letter code provided in the table below.

TABLE 1

| Three-Letter | One-Letter | |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Fexapotide Triflutate (“FT”), as it is used herein, denotes a 17-mer peptide having the amino acid sequence: Ile-Asp-Gln-Gln-Val-Leu-Ser-Arg-lle-Lys-Leu-Glu-lle-Lys-Arg-Cys-Leu (SEQ ID NO. 1). FT is disclosed in U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031. The disclosures of these patents and published applications are incorporated by reference herein in their entirety. FT is represented by SEQ ID NO. 1, shown below:

SEQ ID NO. 1:
IDQQVLSRIKLEIKRCL
or
Ile-Asp-Gln-Gln-Val-Leu-Ser-Arg-Ile-Lys-
Leu-Glu-Ile-Lys-Arg-Cys-Leu.

The term “fragment” refers to a protein or polypeptide that consists of a continuous subsequence of the amino acid sequence of a protein or peptide and includes naturally occurring fragments such as splice variants and fragments resulting from naturally occurring in vivo protease activity. Such a fragment may be truncated at the amino terminus, the carboxy terminus, and/or internally (such as by natural splicing). Such fragments may be prepared with or without an amino terminal methionine. The term “fragment” includes fragments, whether identical or different, from the same protein or peptide, with a contiguous amino acid sequence in common or not, joined together, either directly or through a linker. A person having ordinary skill in the art will be capable of selecting a suitable fragment for use in the embodiments without undue experimentation using the guidelines and procedures outlined herein.

The term “variant” refers to a protein or polypeptide in which one or more amino acid substitutions, deletions, and/or insertions are present as compared to the amino acid sequence of an protein or peptide and includes naturally occurring allelic variants or alternative splice variants of an protein or peptide. The term “variant” includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.) Preferred variants include alanine substitutions at one or more of amino acid positions. Other preferred substitutions include conservative substitutions that have little or no effect on the overall net charge, polarity, or hydrophobicity of the protein. Conservative substitutions are set forth in Table 2 below.

TABLE 2
Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

Table 3 sets out another scheme of amino acid substitution:

TABLE 3

| Original Residue | Substitutions |
|---|---|
| Ala | gly; ser |
| Arg | lys |
| Asn | gln; his |
| Asp | glu |
| Cys | ser |
| Gln | asn |
| Glu | asp |
| Gly | ala; pro |
| His | asn; gln |
| Ile | eu; val |
| Leu | ile; val |
| Lys | arg; gln; glu |
| Met | leu; tyr; ile |
| Phe | met; leu; tyr |
| Ser | thr |
| Thr | ser |
| Trp | tyr |
| Tyr | trp; phe |
| Val | ile; leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine. Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include proteins and peptides with additional amino acid residues before or after the protein or peptide amino acid sequence on linker peptides. For example, a cysteine residue may be added at both the amino and carboxy terminals of FT in order to allow the cyclisation of the peptide by the formation of a di-sulphide bond. The term “variant” also encompasses polypeptides that have the amino acid sequence of FT with at least one and up to 25 or more additional amino acids flanking either the 3′ or 5′ end of the peptide.

The term “derivative” refers to a chemically modified protein or polypeptide that has been chemically modified either by natural processes, such as processing and other post-translational modifications, but also by chemical modification techniques, as for example, by addition of one or more polyethylene glycol molecules, sugars, phosphates, and/or other such molecules, where the molecule or molecules are not naturally attached to wild-type proteins or FT. Derivatives include salts. Such chemical modifications are well described in basic texts and in more detailed monographs, as well as in a voluminous research literature, and they are well known to those of skill in the art. It will be appreciated that the same type of modification may be present in the same or varying degree at several sites in a given protein or polypeptide. Also, a given protein or polypeptide may contain many types of modifications. Modifications can occur anywhere in a protein or polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2nd Ed., T. E. Creighton, W. H. Freeman and Company, New York (1993) and Wold, F., “Posttranslational Protein Modifications: Perspectives and Prospects,” pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., “Protein Synthesis: Posttranslational Modifications and Aging,” Ann. N.Y. Acad. Sci. 663: 48-62 (1992). The term “derivatives” include chemical modifications resulting in the protein or polypeptide becoming branched or cyclic, with or without branching. Cyclic, branched and branched circular proteins or polypeptides may result from post-translational natural processes and may be made by entirely synthetic methods, as well.

The term “homologue” refers to a protein that is at least 60 percent identical in its amino acid sequence of FT as determined by standard methods that are commonly used to compare the similarity in position of the amino acids of two polypeptides. The degree of similarity or identity between two proteins can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carillo H. and Lipman, D., SIAM, J. Applied Math., 48:1073 (1988). Preferred methods to determine identity are designed to give the largest match between the sequences tested. Methods to determine identity and similarity are codified in publicly available computer programs.

Preferred computer program methods useful in determining the identity and similarity between two sequences include, but are not limited to, the GCG program package (Devereux, J., et al., Nucleic Acids Research, 12(1): 387 (1984)), BLASTP, BLASTN, and FASTA, Atschul, S. F. et al., J. Molec. Biol., 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol., 215: 403-410 (1990). By way of example, using a computer algorithm such as GAP (Genetic Computer Group, University of Wisconsin, Madison, Wis.), the two proteins or polypeptides for which the percent sequence identity is to be determined are aligned for optimal matching of their respective amino acids (the “matched span”, as determined by the algorithm).

A gap opening penalty (which is calculated as 3 times the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al. in: Atlas of Protein Sequence and Structure, vol. 5, supp.3 for the PAM250 comparison matrix; see Henikoff et al., Proc. Natl. Acad. Sci USA, 89:10915-10919 for the BLOSUM 62 comparison matrix) also may be used by the algorithm. The percent identity then is calculated by the algorithm. Homologues will typically have one or more amino acid substitutions, deletions, and/or insertions as compared with the comparison protein or peptide, as the case may be.

The term “fusion protein” refers to a protein where one or more peptides are recombinantly fused or chemically conjugated (including covalently and non-covalently) to a protein such as (but not limited to) an antibody or antibody fragment like an Fab fragment or short chain Fv. The term “fusion protein” also refers to multimers (i.e. dimers, trimers, tetramers and higher multimers) of peptides. Such multimers comprise homomeric multimers comprising one peptide, heteromeric multimers comprising more than one peptide, and heteromeric multimers comprising at least one peptide and at least one other protein. Such multimers may be the result of hydrophobic, hyrdrophilic, ionic and/or covalent associations, bonds or links, may be formed by cross-links using linker molecules or may be linked indirectly by, for example, liposome formation.

The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. Examples of peptide mimetics in this broader sense (where part of a peptide is replaced by a structure lacking peptide bonds) are described below. Whether completely or partially non-peptide, peptide mimetics according to the embodiments provide a spatial arrangement of reactive chemical moieties that closely resemble the three-dimensional arrangement of active groups in the peptide on which the peptide mimetic is based. As a result of this similar active-site geometry, the peptide mimetic has effects on biological systems that are similar to the biological activity of the peptide.

The peptide mimetics of the embodiments are preferably substantially similar in both three-dimensional shape and biological activity to the peptides described herein. Examples of methods of structurally modifying a peptide known in the art to create a peptide mimetic include the inversion of backbone chiral centers leading to D-amino acid residue structures that may, particularly at the N-terminus, lead to enhanced stability for proteolytical degradation without adversely affecting activity. An example is given in the paper “Tritriated D-ala.sup. 1-Peptide T Binding”, Smith C. S. et al., Drug Development Res., 15, pp. 371-379 (1988). A second method is altering cyclic structure for stability, such as N to C interchain imides and lactames (Ede et al. in Smith and Rivier (Eds.) “Peptides: Chemistry and Biology”, Escom, Leiden (1991), pp. 268-270). An example of this is given in conformationally restricted thymopentin-like compounds, such as those disclosed in U.S. Pat. No. 4,457,489 (1985), Goldstein, G. et al., the disclosure of which is incorporated by reference herein in its entirety. A third method is to substitute peptide bonds in the peptide by pseudopeptide bonds that confer resistance to proteolysis.

A number of pseudopeptide bonds have been described that in general do not affect peptide structure and biological activity. One example of this approach is to substitute retro-inverso pseudopeptide bonds (“Biologically active retroinverso analogues of thymopentin”, Sisto A. et al in Rivier, J. E. and Marshall, G. R. (eds) “Peptides, Chemistry, Structure and Biology”, Escom, Leiden (1990), pp. 722-773) and Dalpozzo, et al. (1993), Int. J. Peptide Protein Res., 41:561-566, incorporated herein by reference). According to this modification, the amino acid sequences of the peptides may be identical to the sequences of an peptide described above, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus. Further modifications also can be made by replacing chemical groups of the amino acids with other chemical groups of similar structure. Another suitable pseudopeptide bond that is known to enhance stability to enzymatic cleavage with no or little loss of biological activity is the reduced isostere pseudopeptide bond (Couder, et al. (1993), Int. J. Peptide Protein Res., 41:181-184, incorporated herein by reference in its entirety).

Thus, the amino acid sequences of these peptides may be otherwise identical to the sequence of FT, except that one or more of the peptide bonds are replaced by an isostere pseudopeptide bond. Preferably the most N-terminal peptide bond is substituted, since such a substitution would confer resistance to proteolysis by exopeptidases acting on the N-terminus. The synthesis of peptides with one or more reduced isostere pseudopeptide bonds is known in the art (Couder, et al. (1993), cited above). Other examples include the introduction of ketomethylene or methylsulfide bonds to replace peptide bonds.

Peptoid derivatives of peptides represent another class of peptide mimetics that retain the important structural determinants for biological activity, yet eliminate the peptide bonds, thereby conferring resistance to proteolysis (Simon, et al., 1992, Proc. Natl. Acad. Sci. USA, 89:9367-9371, incorporated herein by reference in its entirety). Peptoids are oligomers of N-substituted glycines. A number of N-alkyl groups have been described, each corresponding to the side chain of a natural amino acid (Simon, et al. (1992), cited above). Some or all of the amino acids of the peptides may be replaced with the N-substituted glycine corresponding to the replaced amino acid.

The term “peptide mimetic” or “mimetic” also includes reverse-D peptides and enantiomers as defined below.

The term “reverse-D peptide” refers to a biologically active protein or peptide consisting of D-amino acids arranged in a reverse order as compared to the L-amino acid sequence of an peptide. Thus, the carboxy terminal residue of an L-amino acid peptide becomes the amino terminal for the D-amino acid peptide and so forth. For example, the peptide, ETESH, becomes H_{d}S_{d}E_{d}T_{d}E_{d}, where E_{d}, H_{d}, S_{d}, and T_{d} are the D-amino acids corresponding to the L-amino acids, E, H, S, and T respectively.

The term “enantiomer” refers to a biologically active protein or peptide where one or more the L-amino acid residues in the amino acid sequence of an peptide is replaced with the corresponding D-amino acid residue(s).

A “composition” as used herein, refers broadly to any composition containing FT and, optionally an additional active agent. The composition may comprise a dry formulation, an aqueous solution, or a sterile composition. Compositions comprising FT may be employed as hybridization probes. The probes may be stored in freeze-dried form and may be associated with a stabilizing agent such as a carbohydrate. In hybridizations, the probe may be deployed in an aqueous solution containing salts, e.g., NaCl, detergents, e.g., sodium dodecyl sulfate (SDS), and other components, e.g., Denhardt's solution, dry milk, salmon sperm DNA, etc.

In an embodiment, the expression “alpha1-adrenergic receptor blocker(s)”, or “alpha blocker” denotes one or more selected from the group consisting of tamsulosin, terazosin, doxazosin, prazosin, bunazosin, indoramin, alfulzosin, and silodosin. In an embodiment, the expression “5 α-reductase inhibitor(s)” or “5-ARI” denotes one or more selected from the group consisting of finasteride and dutasteride. In another embodiment in which an additional active agent is used together with FT, the 5-ARI and the alpha blocker, the expression “active agent” is used to denote any agent that provides a therapeutic effect to a subject in need, and preferably is an agent capable of removing unwanted cellular proliferations and/or tissue growth. Suitable active agents may include, but are not limited to: (i) anti-cancer active agents (such as alkylating agents, topoisomerase I inhibitors, topoisomerase II inhibitors, RNA/DNA antimetabolites, and antimitotic agents); (ii) active agents for treating benign growths such as anti-acne and anti-wart active agents; (iii) antiandrogen compounds, (cyproterone acetate (1α, 2β-methylene-6-chloro-17 α-acetoxy-6-dehydroprogesterone) Tamoxifen, aromatase inhibitors); (iv) phosphodiesterase type 5 (PDE5) inhibitors (tadalafil), and combinations thereof.

Throughout this disclosure, the term “mean IPSS” denotes the mean of all of the test subject's total IPSS scores, and the expression “mean IPSS improvement” when reported as a number, denotes the mean IPSS improvement measured at a period of time after administration of a control or test therapy, when compared to the mean IPSS measured before administration of a control or test therapy (referred to as “baseline IPSS”). Improvement in IPSS is denoted by a lower number, and hence the expression “mean IPSS improvement” represents the decrease in mean IPSS from mean baseline IPSS.

The expression “relative mean IPSS improvement” denotes the percentage difference between two variables and is determined as the relative change from variable 1 to variable 2. For example, assume the mean IPSS improvement for doxazosin alone is 6.6, the mean IPSS improvement for finasteride alone is 5.6, and the mean IPSS improvement for the combination is 7.4. The average mean IPSS improvement for each active alone is about 6.1, ((5.6+6.6)/2). The relative mean IPSS improvement for the combination, when compared to the average mean IPSS improvement for each active alone therefore is simply the percentage difference between 7.4 and 6.1, or about 21.3% ((7.4-6.1)/6.1).

The embodiments include compositions and methods that utilize a specific peptide described by the amino acid sequence lle-Asp-Gln-Gln-Val-Leu-Ser-Arg-lle-Lys-Leu-Glu-lle-Lys-Arg-Cys-Leu, (Fexapotide Triflutate or “FT”), combined with at least one 5 α-reductase inhibitor (5-ARI) and at least one alpha1-adrenergic receptor blocker (alpha blocker). The inventor discovered that this particular combination of BPH therapies provided an unexpectedly superior improvement in treating BPH, as measured by a decrease in mean IPSS scores, when compared to the improvement found from either therapy alone, the average improvement of the therapies, and the improvement from the average of (a) the improvement from FT and (b) the improvement from the combination of at least one 5-ARI and at least one alpha blocker.

The embodiments also include a combination of BPH therapies that includes a composition comprising FT and a pharmaceutically acceptable carrier, a composition comprising at least one 5-ARI and a pharmaceutically acceptable carrier, and a composition comprising at least one alpha blocker and a pharmaceutically acceptable carrier. Alternatively, the 5-ARI and alpha blocker may be combined in a single composition, also including a pharmaceutically acceptable carrier. In one embodiment, the composition comprising FT is an injectable composition that is administered once, or administered more than once with a period of time between each administration of from about 1 month to about 3 years, and the compositions comprising the 5-ARI and alpha blocker, or a composition comprising a combination of a 5-ARI and an alpha blocker, are compositions to be administered orally and taken daily, or intermittently over a period of time.

The composition comprising FT can be administered intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transrectally, transperitoneally, transdermally, via an aerosol, infusion, bolus injection, implantation device, sustained release system etc. Alternatively, FT can be expressed in vivo by administering a gene that expresses FT, by administering a vaccine that induces such production or by introducing cells, bacteria or viruses that express FT in vivo, because of genetic modification or otherwise. In an embodiment, the compositions comprising the 5-ARI and alpha blocker, or a single composition comprising a combination of a 5-ARI and an alpha blocker, can be administered together or separately from the composition comprising FT or the agent that expresses FT in vivo.

Another embodiment includes a method of treating BPH by administering a first composition comprising a therapeutically effective amount of FT; and subsequently orally administering a combination of a 5-ARI and an alpha blocker present in a single oral composition, or present in two separate oral compositions. In accordance with this method, the relative mean IPSS improvement of the combination may be from about 40% to about 100%, when compared to the average median IPSS improvement of all three actives alone, and the relative mean IPSS improvement of the combination® may be from about 45% to about 400%, when compared to the average mean IPSS improvement of (a) FT alone and (b) the combination of a 5-ARI and an alpha blocker.

Suitable 5α-reductase inhibitors (5-ARIs) include those that block the action of the 5α-reductase enzymes that convert testosterone into DHT. One such 5-ARI is 17β-N-(2,5-bis(Trifluoromethyl))phenylcarbamoyl-4-aza-5α-androst-1-en-3-one, known as dutasteride, and sold under the brand name Avodart®. This compound has been reported to inhibit all three forms of 5α-reductase, and can decrease DHT levels in the blood by up to 98%. Specifically it is a competitive, mechanism-based (irreversible) inhibitor of all three isoforms of 5α-reductase, types I, II, and III. The activity of dutasteride is in contrast to finasteride, which is similarly an irreversible inhibitor of 5α-reductase, but only inhibits the type II and Illisoenzymes. As a result of this difference, dutasteride is able to achieve a reduction in circulating DHT levels of up to 98%, whereas finasteride is able to achieve a reduction of only 65 to 70%. In spite of the differential reduction in circulating DHT levels, the two drugs decrease levels of DHT to a similar extent of approximately 85 to 90% in the prostate gland, where the type II isoform of 5α-reductase predominates, and thus are considered equally effective in treating BPH.

Alpha₁-adrenergic receptor blockers (“alpha blockers”) function generally as anti-hypertensive agents by blocking α-adrenergic receptor sites. They relax stromal (smooth) tissue in the bladder, which cause fibrous tissue to contract when stimulated by noradrenaline and results in decreased urinary flow rates. Thus, the effect of the α₁-blocker is to relax the fibrous tissue and result in increased urinary flow rates. α-Adrenergic blocking agents bind selectively to the α class of adrenergic receptors and thereby interfere with the capacity of sympathomimetic amines to initiate actions at these sites.

There are prominent differences in the relative abilities of α-adrenergic blocking agents to antagonize the effects of sympathomemetic amines at the two subtypes of a receptors. It is known that Prazosin is much more potent in blocking a₁-(postsynaptic) receptors than α₂-receptors that, among other effects, modulate neural release of transmitter (presumed presynaptic receptors). Phenoxybenzamine is a moderately selective unblocking agent, while phentolamine is only three to five times more potent in inhibiting α₁- than α₂-adrenergic receptors. In contrast, yohimbine is a selective α₂-blocker and has been shown to prevent the antihypertensive effects of clonidine, an α₂-agonist.

An embodiment includes α₁-adrenergic blockers that have little or no α₂-blocking activity. Examples of such α-adrenergic receptor blockers are terazosin (Abbott-Hytrin*) whose chemical name is 1-(4-amino-6,7-dimethoxy-2-quinazolinyl)-4-[(tetrahydro-2-furanyl) carbonyl]piperazine, as described in German Patent 2,646,186 and U.S. Pat. No. 4,026,894; doxazosin (Pfizer-Cardura*) whose chemical name is 1-(4-amino-6,7-dimethoxy-2-quinazolinyl)-4-[(2,3-dihydro-1,4-benzodioxin-2-yl)carbonyl]piperazine, as described in German Patent 2,847,623 and U.S. Pat. No. 4,188,390; prazosin (PfizerMinipres*) whose chemical name is 1-(4-amino-6,7-dimethoxy-2-quinazolinyl)-4-(2-furanylcarbonyl) piperazine, as described in British Patent 1,156,973, U.S. Pat. No. 3,511,836 and Netherlands Patent Appln. 7,206,067; tamsulosin, whose chemical name is (R)-5-(2-{[2-(2-Ethoxyphenoxy)ethyl]amino}propyl)-2-methoxybenzene-1-sulfonamide, as described in U.S. Pat. No. 4,703,063; bunazosin (Sandoz-Detantol*) whose chemical name is 1-(4-amino-6,7-dimethoxy-2-quinazolinyl)hexahydro 4-(1-oxobutyl)-1H-1,4-diazepine, as described in Belgian Pat. Appln. 806,626, U.S. Pat. No. 3,920,636 and Japanese Kokai 75,140,474; indoramin (Baratol*-Wyeth) whose chemical name is N-[1-[2-(1H-indol-3-yl)ethyl]-4-piperidinyl]benzamide, as described in South African Patent 68 03204, U.S. Pat. No. 3,527,761; Alfuzosin (Synthelabs) whose chemical name is N-[3-[(4-amino-6,7-dimethoxy-2-quinazolinyl)methylamino]propyl] tetrahydro-2-furancarboxamide, as described in German Patent 2,904,445 and U.S. Pat. No. 4,315,007.

Combinations of FT and conventional oral medications are described in, for example, U.S. Patent Application Publication No. 2016/0361,380, an application filed by the present inventor, the disclosure of which is incorporated by reference herein in its entirety. The application discloses the combination of FT with conventional oral medications for BPH, and lists a number of 5-ARIs, alpha blockers, PDE5 inhibitors, or combinations thereof, but does not disclose in its examples a combination of FT, a 5-ARI, and an alpha blocker (see paragraph of U.S. Patent Application Publication No. 2016/0361,380). Based on the disclosure of this application, a person having ordinary skill in the art would have reasonably expected that combinations of FT with any of the listed agents, or combinations of those agents, would provide the same or similar results. The present inventor unexpectedly discovered when treating mammals with BPH who had not previously taken oral medications for BPH, however, that combinations of FT and PDE5 inhibitors such as tadalafil were ineffective, and that the triple combination of FT, a 5-ARI, and an alpha blocker produced an improvement that was significantly greater than the improvement from the combination of FT and a 5-ARI, or the combination of FT and an alpha blocker.

The inventor unexpectedly discovered that administration of FT by intraprostatic injection to a mammal having BPH, and that had not previously taken oral medications for BPH, and then administration to the mammal a combination of a 5-ARI and an alpha blocker, significantly improved the symptoms of BPH, as determined by the IPSS. In one embodiment, administration of the combination of a 5-ARI and an alpha blocker takes place from about 2 months to about 5 years after administration of FT, or from about 2 months to about 4 years, or from about 3 months to about 3 years, or from about 1 year to about 3 years, or any value in between. While not intending on being bound by any particular theory or operation, it is believed possible that the delay between administration of FT and administration of the combination of 5-ARI and alpha blocker may provide a “boosting” effect insofar as all three actives have different mechanisms of action.

In an embodiment, the method includes identifying a mammal having BPH, who had not previously taken an oral medication for BPH, administering to the mammal a composition comprising a therapeutically effective amount of FT, and within a period of from about 2 months to about 5 years, administering to the mammal a combination of a 5-ARI and an alpha blocker. In an embodiment, the mammals having BPH who had not previously taken an oral medication for BPH were administered a course of antibiotics beginning from about 2 to about 4 weeks prior to administration of the composition comprising a therapeutically effective amount of FT. Any antibiotic, or combinations thereof, may be used in the embodiments. It is preferred that the antibiotic is one effective in preventing or reducing the incidence of bacterial infection that may be associated with urinary tract infections. Antibiotics used should provide adequate protection against the commonly encountered bacterial strains of uropathogens including: Escherichia coli, Streptococcus faecalis, Proteus/Pseudomonas spp. and coagulase-positive Staphylococcus. In an embodiment, the method encompasses administration of one, two, three or more antibiotics in the same or different formulation, and by the same or different administrative route. Antibiotics used in the embodiments may be selected from one or more of erythromycin, kitasamycin, streptomycin cephalothin, cephazolin, tetracycline, gramicidin, griseofulvin, gentamicin, novobiocin, ampicillin, imipenem, metronidazole, ceftriaxone, cephalexin, ciprofloxacin, gemifloxacin, fosfomycin, levofloxacin, moxifioxacin, norfloxacin, nitrofurantoin, ofloxacin, trimethoprim/sulfamethoxaxole, and derivatives and salts of any of the foregoing. The antibiotics also may be selected from one or more of ampicillin, gentamicin, imipenem, cephalothin, metronidazole, ciprofloxacin, gemifloxacin, fosfomycin, levofloxacin, moxifloxacin, norfloxacin, nitrofurantoin, and ofloxacin. The antibiotics may be administered in two or three different courses, including a course of a fluoroquinolone antibiotic, a course of metronidazole, and an intramuscular injection of an antibiotic selected from imepenem, gentamicin, and cephalothin.

In an embodiment, the effectiveness of administering the combination of BPH therapies is determined by assessing the mean IPSS anywhere from 3 months to 4 years after administration of the combination of 5-ARI and alpha blocker, (which as described above, the combination of 5-ARI and alpha blocker is administered a period of time after administration of FT), and calculating the improvement relative to the average of the mean IPSS improvements of the individual therapies, where the mean IPSS improvement for each treatment is determined relative to a control. The mean IPSS improvement for individual therapies (or monotherapy, or combination therapy in the case of a combination of a 5-ARI and an alpha blocker), also should be assessed anywhere from 3 months to 4 years after administration of the monotherapy. It is beneficial if the difference in time between: (a) when the mean IPSS of the combination of the embodiments is assessed; and (b) when the mean IPSS for the monotherapies are assessed, is less than 20 months, or less than 15 months, or less than 10 months, or between about 2 months and about 7 months. It also is beneficial if the mean IPSS for all of the studies is assessed at or about the same period of time from when the mammal was first administered either a placebo or a compositions comprising FT. In one embodiment, that period of time is from about 2 years to about 8 years after the first administration, (or trial initiation), or from about 2.5 to about 7 years, or from about 3 to about 6 years, or at about 4 years. In an embodiment, the mean IPSS for the combination of FT, 5-ARI, and alpha blocker is assessed anywhere from about 6 months to 2 years after administration of the combination of 5-ARI and alpha blocker, or from about 10 months to about 2 years, or from about one year to two years, or any value therebetween. In another embodiment, the mean IPSS improvement for individual therapies (or monotherapy, or combination therapy in the case of a combination of a 5-ARI and an alpha blocker), is assessed anywhere from 6 months to 2 years after administration of the monotherapy (or combination therapy in the case of a combination of a 5-ARI and an alpha blocker), or from about 10 months to about 2 years, or from about one year to two years, or any value therebetween.

For a triple combination, the effectiveness can be measured by determining the improvement relative to the average improvement of one of the actives and a combination of the other two actives (e.g., the average of the improvements of (a) 1 alone, and (b) the improvement of the combination of 2 and 3). The effectiveness also can be measured by determining the improvement of the triple combination relative to the average of each active alone (or average of the monotherapy improvements), where the improvement is measured relative to the IPSS measured at baseline. It was known that combinations of medications, even those having the same or similar mechanism of action, are rarely if ever, purely additive, when used in a clinical setting. In other words, a person having ordinary skill in the art would not have expected a combination of BPH medications to have a purely additive effect where the effects of each individual medication can simply be added together to provide a combined effect. This is shown in the results of combination BPH therapies that were published by Miller and McConnell discussed in the Background section above.

For exemplary purposes only, assume that the mean IPSS at baseline is the same for all mammals, such as about 23.5 (a score of above 20 is typically indicative of severe symptoms of BPH). About 2 years after administration of FT, the mean IPSS was 18.3, resulting in a mean IPSS improvement of 5.2 (23.5-18.3). About 2 years after administration of a control, compositions comprising a 5-ARI, an alpha blocker, tadalafil, or a combination of a 5-ARI and an alpha blocker were administered, and about 2 years after that, the mean IPSS for the 5-ARI was 17.3, the mean IPSS for the alpha blocker was 17.91, the mean IPSS for tadalafil was 18.83, and the mean IPSS for the combination of a 5-ARI and an alpha blocker was 23.07. The respective mean IPSS improvements therefore were 6.2 for the 5-ARI, 5.59 for the alpha blocker, 4.67 for the PDE5 inhibitor, and 0.43 for the combination of a 5-ARI and an alpha blocker. The mean IPSS improvement for all of the respective monotherapies therefore have been determined. Continuing with the exemplary calculation, about 2.5 years after administration of FT, a combination of a 5-ARI and an alpha blocker was administered, and about 15 months later the mean IPSS of the triple combination was assessed as 13.03, providing a mean IPSS improvement of 10.47 for the triple combination. The effectiveness of this combination therefore can be determined relative to the average of all three monotherapies (the average of FT (5.2), 5-ARI (6.2), and the alpha blocker (5.59)), which is 5.66, and determined by the relative change ((10.47-5.66)/5.66), which is about 85%. Alternatively, the percent improvement of the triple combination when compared to the average of (a) FT monotherapy, and (b) a 5-ARI and alpha blocker combination therapy can be determined by the relative change ((10.47-2.82)/2.82), which is about 271%.

In accordance with another embodiment, the inventor discovered that methods described provided a relative mean IPSS improvement of the combination may be from about 40% to about 100%, when compared to the average median IPSS improvement of all three actives alone, and the relative mean IPSS improvement of the combination may be from about 45% to about 400%, when compared to the average mean IPSS improvement of (a) FT alone and (b) the combination of a 5-ARI and an alpha blocker. In an embodiment, methods described herein provide a relative mean IPSS improvement of the combination of from about 50% to about 95%, or from about 65% to about 90% or from about 70% to about 87%, or any value therebetween, when compared to the average median IPSS improvement of all three actives alone. In an embodiment, methods described herein provide a relative mean IPSS improvement of the combination of from about 75% to about 350%, or from about 85% to about 300%, or from about 100% to about 300%, when compared to the average mean IPSS improvement of (a) FT alone and (b) the combination of a 5-ARI and an alpha blocker.

Any mammal can benefit from use of the invention, including humans, mice, rabbits, dogs, sheep and other livestock, any mammal treated or treatable by a veterinarian, zoo-keeper, or wildlife preserve employee. Preferred mammals are humans, sheep, and dogs. Throughout this description mammals and patients are used interchangeably.

It will be apparent to one of skill in the art that other smaller fragments of FT may be selected such that these peptides will possess the same or similar biological activity. Other fragments of FT may be selected by one skilled in the art such that these peptides will possess the same or similar biological activity. The term “FT” as used in the embodiments therefore encompasses these other fragments. In general, the peptides of the embodiments have at least 4 amino acids, preferably at least 5 amino acids, and more preferably at least 6 amino acids.

The embodiments also encompass methods of treatment comprising administering a composition comprising FT that includes two or more FT sequences joined together, together with an additional active agent. To the extent that FT has the desired biological activity, it follows that two or more FT sequences would also possess the desired biological activity.

FT and fragments, variants, derivatives, homologues, fusion proteins and mimetics thereof encompassed by this embodiment can be prepared using methods known to those of skill in the art, such as recombinant DNA technology, protein synthesis and isolation of naturally occurring peptides, proteins, variants, derivatives and homologues thereof. FT and fragments, variants, derivatives, homologues, fusion proteins and mimetics thereof can be prepared from other peptides, proteins, and fragments, variants, derivatives and homologues thereof using methods known to those having skill in the art. Such methods include (but are not limited to) the use of proteases to cleave the peptide, or protein into FT. Any method disclosed in, for example, U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, can be used to prepare the FT peptide described herein. The disclosures of these patent documents are incorporated by reference herein in their entireties.

Therapeutic compositions described herein may comprise an amount of FT in admixture with a pharmaceutically acceptable carrier. In some alternative embodiments, the 5-ARI and alpha blocker can be administered in the same composition with FT, and in other embodiments, the composition comprising FT is administered as an injection, whereas the 5-ARI and alpha blockers are formulated into an oral medication (gel, capsule, tablet, liquid, etc.), either as two separate oral medications, or a combined oral medication. The carrier material may be water or phosphate buffered saline for injection, preferably supplemented with other materials common in solutions for administration to mammals. Typically, FT will be administered in the form of a composition comprising the purified FT peptide (or chemically synthesized FT peptide) in conjunction with one or more physiologically acceptable carriers, excipients, or diluents. Neutral buffered saline or saline mixed with serum albumin are exemplary appropriate carriers. Preferably, the product is formulated as a lyophilizate using appropriate excipients (e.g., sucrose). Other standard carriers, diluents, and excipients may be included as desired. Compositions of the embodiments also may comprise buffers known to those having ordinary skill in the art with an appropriate range of pH values, including Tris buffer of about pH 7.0-8.5, or acetate buffer of about pH 4.0-5.5, which may further include sorbitol or a suitable substitute therefor.

Solid dosage forms for oral administration include but are not limited to, capsules, tablets, pills, powders, and granules. In such solid dosage forms, the 5-ARI, the alpha blocker, and/or FT can be admixed with at least one of the following: (a) one or more inert excipients (or carrier), such as sodium citrate or dicalcium phosphate; (b) fillers or extenders, such as starches, lactose, sucrose, glucose, mannitol, and silicic acid; (c) binders, such as carboxymethylcellulose, alginates, gelatin, polyvinylpyrrolidone, sucrose and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as acetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the active compounds, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Actual dosage levels of the respective active ingredients in the compositions of the embodiments may be varied to obtain amounts of FT, 5-ARI, and alpha blocker that are effective to obtain a desired therapeutic response for a particular composition. The selected dosage level therefore depends upon the desired therapeutic effect, the route of administration, the desired duration of treatment, the weight of the mammal, the severity of BPH, and other factors.

With mammals, including humans, the effective amounts can be administered on the basis of body surface area. The interrelationship of dosages for animals of various sizes, species and humans (based on mg/M2 of body surface) is described by E. J. Freireich et al., Cancer Chemother. Rep., 50 (4):219 (1966). Body surface area may be approximately determined from the height and weight of an individual (see e.g., Scientific Tables, Geigy Pharmaceuticals, Ardsley, N.Y. pp. 537-538 (1970)).

It will be understood that the specific dose level for any particular patient will depend upon a variety of factors including the body weight, general health, sex, diet, time and route of administration, potency of the administered drug, rates of absorption and excretion, combination with other drugs and the severity of the particular disease being treated. In an embodiment, FT is present in an injectable composition in an amount ranging from about 0.5 mg to about 350 mg, or from about 1.0 mg to about 100 mg, or from about 2.0 mg to about 25 mg, or about 2.5 mg, or any value in between these ranges.

In an embodiment, the 5-ARI is present in the oral medication in an amount of from about 0.1 mg to about 10 mg, or from about 0.25 to about 5 mg, or from about 1 to about 5 mg, or about 0.5 mg, or any value therebetween. The alpha blocker also may be present in the same oral medication as the 5-ARI, or a separate oral medication, and may be present in an amount of from about 0.1 mg to about 20 mg, or from about 0.25 to about 15 mg, or from about 0.5 to about 10 mg, or about 0.5 mg, or any value therebetween. In one exemplary embodiment, the composition comprising FT is injected once via intraprostatic injection, and at least 3 months later, the 5-ARI and the alpha blocker are taken orally, once daily. In another embodiment, a composition comprising FT is injected via intraprostatic injection at least one additional time at a period of time at least 2 to 12 months after the initial injection.

A method of administering a composition comprising FT according to the embodiments includes, but is not limited to, administering the compositions intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transrectally, transperitoneally, transdermally, via an aerosol, infusion, bolus injection, implantation device, sustained release system etc. Any method of administration disclosed in, for example, U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, can be used.

FT is a new molecular entity which in vitro stimulates caspase pathways (activation of caspases 7, 8, and 10, caspase recruitment domains 6, 11, and 14, and DIABLO), tumor necrosis factor pathways (activation of TNF1, TNFSF6, TNFSF8, TNFSF9, CD70 ligands, and TNFRSF19L, TNFRSF25, TRAF2, TRAF3, TRAF4, TRAF6 receptors), and BCL pathways (activation of BIK, HRK, BCL2L10 and BCL3) in prostate glandular epithelial cells, based on tissue culture genetic array data. FT selectively causes loss of cell membrane integrity, mitochondrial metabolic arrest, depletion of RNA, DNA lysis and aggregation, and cell fragmentation and cell loss. The apoptotic process leads to typical ultrastructural progressive changes of membranous disruption and swelling, progressively deepening nuclear invaginations with eventual membranous bleb formations and cell death and fragmentation into apoptotic bodies. Histologically, typical apoptotic changes with positive immunohistochemical staining of markers for apoptosis are found throughout the injected areas for up to several weeks after treatment.

FT has been extensively tested in patients with BPH and in men with low-grade (T1c) prostate cancer. The compound and placebo controls have been administered by the transrectal route in over 1700 procedures in 9 human clinical trials. In these large long-term clinical trials in men with BPH, FT was administered in a concentration of 0.25 mg/ml (2.5 mg of FT). See, e.g., Shore, et al., “The potential for NX-1207 in benign prostatic hyperplasia: an update for clinicians,” Ther Adv. Chronic Dis., 2(6), pp. 377-383 (2011).

The following examples are provided to illustrate the present embodiments. It should be understood, however, that the embodiments are not to be limited to the specific conditions or details described in these examples. Throughout the specification, any and all references to a publicly available document, including a U.S. patent, are specifically incorporated by reference. In particular, the embodiments expressly incorporate by reference the examples contained in U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, each of which reveal that certain peptides specified therein are effective agents for causing cell death in vivo in normal rodent muscle tissue, subcutaneous connective tissue, dermis and other tissue.

### Examples

Clinical trials were conducted on numerous individuals having BPH. All protocols were done in accordance with applicable regulations, and carried out by physicians.

In a study of 978 men, patients with BPH were first administered a course of antibiotics for a period of 2-4 weeks, and then given an intraprostatic injection of either a) 10 ml of FT in phosphate buffered saline (concentration of 0.25 mg/ml), pH 7.2 (“PBS”) or b) PBS alone, under double-blind conditions by a urologist in an office setting under ultrasound guidance. Each patient was followed for a number of years with regular physical examinations, laboratory tests, and evaluations of symptoms. Symptomatic evaluation was measured by the International Prostate Symptom Score (IPSS) which is a quantitative scale used to gauge prostatic symptomatic improvement or worsening. The IPSS quantifies the following: 1) incomplete bladder emptying after urination; 2) frequent urination; 3) stopping and starting during urination; 4) urgent need to urinate; 5) weakness of urinary stream; 6) need to push or strain during urination; 7) need to urinate after going to sleep at night (nocturia). The difference from baseline IPSS at an average of 22 months after treatment was compared in patients who were given FT vs patients who received PBS alone. In order to determine the effect of FT alone, patients who had no previous history of conventional drug treatment (“treatment naïve”) were administered FT and compared to treatment naïve patients who only received a control. Approximately 359 patients were excluded because they had received conventional oral drug treatment either before or during the assessment. Other patients were only followed for 12 months and were not included. The data are shown in Table 4 below.

TABLE 4

| | | | Duration | |
|---|---|---|---|---|
| | | | After | Mean IPSS |
| Group | N | Treatment | Treatment | Improvement |
| No previous treatment | 227 | FT | 22 months | 5.2 * |
| (treatment naive) | | | | |
| No previous treatment | 165 | PBS | 22 months | 3.4 |
| (treatment naive) | | | | |

* 2 sided t test p < .01 compared to PBS group.

Table 4 reveals that use of FT alone in treating BPH provided a Mean IPSS Improvement of about 5.2. To assess the efficacy of conventional oral medications alone, patients who were administered the oral medications listed in the table below, and combinations thereof, together with the same placebo PBS used to assess the efficacy of FT alone, were followed for more than 2 years, and their mean IPSS values were determined. The results are shown in Table 5 below:

TABLE 5

| | | Duration | |
|---|---|---|---|
| | | After | Mean IPSS |
| Group | N | Treatment | Improvement |
| PDE5 Inhibitor (Tadalifil) | 3 | >2 years | 4.67 |
| 5-ARI | 5 | >2 years | 6.2 |
| Alpha Blocker | 27 | >2 years | 5.59 |
| Combination of 5-ARI + | 7 | >2 years | 0.43 |
| Alpha Blocker | | | |

To assess the efficacy of conventional oral medications in combination with FT, patients who were administered FT initially, and then were administered the oral medications listed in Table 5 at a period of time ranging from about 1 year to about 3 years after administration of FT, were followed for more than 2 years, and their mean IPSS values were determined. The results are shown in Table 6 below, together with the IPSS values from Table 5 for the oral medications alone

TABLE 6

| | | | | Mean IPSS | |
|---|---|---|---|---|---|
| | | | | Improvement | |
| | | Duration | | oral | |
| | | After | Mean IPSS | meds alone | |
| Group | N | Treatment | Improvement | (Table 5) | |
| PDE5 Inhibitor + FT | 9 | >2 years | 4.78 | 4.67 | (N = 3) |
| 5-ARI + FT | 3 | >2 years | 9.33 | 6.2 | (N = 5) |
| Alpha Blocker + FT | 41 | >2 years | 8.17 | 5.59 | (N = 27) |
| Combination of 5-ARI + | 15 | >2 years | 10.47 | 0.43 | (N = 7) |
| Alpha Blocker + FT | | | | | |

Using the results from the above tables, the average mean IPSS improvement of the respective medications, or combinations of medications can be determined, as well as the percent improvement in mean IPSS when compared to the average of the actives. The average mean IPSS is simply the average of the mean IPSS values of FT and each of the respective medications. Thus, the average mean IPSS for the PDE5 Inhibitor is simple (5.2+4.67)/2=4.94. The percent improvement for the respective combinations therefore can be determined based on the percent difference between the mean IPSS of the combination and the average mean IPSS of the combination. Thus, the percent improvement for the combination of FT and a PDE5 Inhibitor is the percent difference between 4.78 and 4.94 ((4.78-4.94)/4.78)*100=3.3%. The results are provided in Table 7 below:

TABLE 7

| | | PDE5 | Alpha | | 5-ARI + Alpha | |
|---|---|---|---|---|---|---|
| Medication | FT | Inhibitor | Blocker | 5-ARI | Blocker | |
| Mean IPSS - | 5.2 | 4.67 | 5.59 | 6.2 | 0.43 | (6.2)* |
| Alone | | | | | | |
| Mean IPSS - | — | 4.78 | 8.17 | 9.33 | 10.47 | |
| combined with | | | | | | |
| FT | | | | | | |
| Avg. Mean IPSS | | 4.94 | 5.40 | 5.7 | 2.82 | (5.7) |
| Percent | | −3.3% | 51.3% | 63.7% | 271% | (83.7 %) |

| | |
|---|---|
| Improvement | |
| Avg. Mean IPSS | 5.66 |
| Improvement for | |
| FT, 5-ARI, and | |
| Alpha Blocker** | |
| Percent | 85% |
| Improvement | |
| over average of | |
| all 3 actives | |

*Mean IPSS of combination of 5-ARI and Alpha Blocker was reported in the literature as 6.2. This was based on the use of a different control than the control used in the present examples. The control used in the examples was an intraprostatic injection of PBS, which was preceded by a 2-4 week course of antibiotics prior to injection for all patients. The values in parenthesis represents the reported mean IPSS, the average of the reported mean IPSS and FT, and the precent improvement from the reported.
**The average mean IPSS of FT, 5-ARI, and Alpha Blocker is calculated simply by adding the mean IPSS improvement for each therapy alone and dividing by 3.

As can be seen from Table 7, the combination of FT and the phosphodiesterase 5 (PDE5) inhibitor tadalafil, a known BPH therapy, was not effective. Combinations of FT and either an alpha blocker or a 5-ARI produced improvements of 51.3% and 63.7%, respectively, which are significantly better than other combinations of BPH therapies described in the literature (the combination of doxazosin and finasteride provided an improvement of about 21.3%; and the combination of tamsulosin and dutasteride provided an improvement of about 34.7%). The triple combination of FT, a 5-ARI, and an alpha blocker, however, unexpectedly provided dramatically improved results, when compared the average mean IPSS improvement of all three medications (85.5%). The combination of FT, a 5-ARI, and an alpha blocker also unexpectedly provided dramatically improved results when compared to the average mean IPSS improvement of the combination of a 5-ARI and an alpha blocker (271%, or 83.7% when using the reported mean IPSS improvement of the combination of 5-ARI and an alpha blocker of 6.2). Regardless of what value is used, the value reported in the literature or the values obtained in the present examples, the combination of FT, a 5-ARI, and an alpha blocker provided an improvement on the order of 85% or higher, when compared to the average improvement for all three actives alone, and when compared to the improvement for the combination of a 5-ARI and an alpha blocker.

What is claimed is:

1. A method of treating a mammal having benign prostatic hyperplasia (BPH) who had not previously taken oral medications for BPH comprising:
Identifying mammals having BPH who had not previously taken oral medications for BPH;
administering by intraprostatic injection a composition comprising Fexapotide Triflutate (FT); and
administering at least once daily a 5 α-reductase inhibitor (5-ARI) and an alpha1-adrenergic receptor blocker (alpha blocker) at a period of time of from about 2 months to about 4 years after administration of the composition comprising FT.

2. The method of claim 1, wherein the composition comprising FT comprises a carrier.

3. The method of claim 1, wherein the method comprises administration of about 10 ml of the composition comprising FT, where FT is present in the composition at a concentration of about 2.5 mg/mL.

4. The method of claim 1, wherein the method comprises administration of from about 0.25 to about 5 mg of the 5-ARI, and from about 0.25 to about 15 mg of the alpha blocker.

5. The method of claim 4, wherein the 5-ARI and the alpha blocker are present in the same composition.

6. The method of claim 1, wherein the 5-ARI is selected from the group consisting of tamsulosin, terazosin, doxazosin, prazosin, bunazosin, indoramin, alfulzosin, and silodosin.

7. The method of claim 6, wherein the 5-ARI is tamsulosin.

8. The method of claim 1, wherein the alpha blocker is selected from dutasteride and finasteride.

9. The method of claim 8, wherein the alpha blocker is dutasteride.

10. The method of claim 1, wherein the method provides a relative mean IPSS improvement, when compared to the average median IPSS improvement of all three actives alone, of from about 40% to about 100%.

11. The method of claim 10, wherein the relative mean IPSS improvement is from about 65% to about 90%.

12. The method of claim 1, wherein the method provides a relative mean IPSS improvement, when compared to the average mean IPSS improvement of (a) FT alone and (b) the combination of a 5-ARI and an alpha blocker, of from about 45% to about 400%.

13. The method of claim 12, wherein the relative mean IPSS improvement is from about 100% to about 300%.

14. A method of treating a mammal having benign prostatic hyperplasia (BPH) who had not previously taken oral medications for BPH comprising:
Identifying mammals having BPH who had not previously taken oral medications for BPH;
administering a course of antibiotics to the identified mammals for at least two weeks;
administering by intraprostatic injection about 10 ml of a composition comprising about 0.25 mg/ml of Fexapotide Triflutate (FT) after administering the course of antibiotics; and
administering at least once daily from about 0.25 to about 5 mg of a 5 α-reductase inhibitor (5-ARI) and from about 0.25 to about 15 mg of an alpha1-adrenergic receptor blocker (alpha blocker) at a period of time from about 2 months to about 4 years after administration of the composition comprising FT.


SEQUENCE LISTING (3 sequences)

SEQ ID NO: 1 - protein, 17 aa, unidentified
IDQQVLSRIKLEIKRCL

SEQ ID NO: 2 - protein, 5 aa, synthetic construct
ETESH

SEQ ID NO: 3 - protein, 5 aa, synthetic construct
  SITE 1..5: note = D-amino acids
HSETE